(19) United States
(12) Patent Application Publication
(10) Pub. No.: US 2015/0367071 A1
(43) Pub. Date: Dec. 24, 2015

(54) SYSTEMS AND METHODS FOR TRACKING AND REVERSING ACCIDENTAL OVERDOSE

(71) Applicant: THE UAB RESEARCH FOUNDATION, Birmingham, AL (US)

(72) Inventors: William Bruce DONNELLAN, Birmingham, AL (US); Stefan C. GRANT, Birmingham, AL (US); Erin Chambliss TANNER, Pelham, AL (US)

(21) Appl. No.: 14/765,549

(22) PCT Filed: Feb. 4, 2014
(86) PCT No.: PCT/US2014/014599

§ 371 Date: Aug. 3, 2015

Related U.S. Application Data
(60) Provisional application No. 61/760,392, filed on Feb. 4, 2013.

(51) Int. Cl. A61M 5/172; A61M 5/14

(52) U.S. Cl. CPC A61M 5/1723; A61M 5/1407; A61M 2005/1405; A61M 2205/18; A61M 2205/50; A61M 2230/005; A61M 2230/42

(57) ABSTRACT

In one embodiment, a system for tracking and reversing accidental overdose includes a patient monitoring system configured to monitor a patient parameter that provides an indication as to whether or not the patient is overdosing, an alert system configured to activate when the patient parameter falls below an established threshold, and a reversal agent delivery system configured to automatically administer a reversing agent that counteracts the effects of an analgesic medication being administered to the patient when the patient parameter falls below the established threshold.

### CROSS-REFERENCE TO RELATED APPLICATION(S)

This application claims priority to co-pending U.S. Provisional Application serial number 61/760,392, filed Feb. 4, 2013, which is hereby incorporated by reference herein in its entirety.

### BACKGROUND

A limiting factor in providing adequate pain control to hospitalized patients is the concern by staff and physicians that an accidental overdose will occur when administering an analgesic medication. In 2004, adverse drug reactions were found in 1,211,100 hospital stays, which accounted for 3.1% of all hospital stays. Of those reactions, drug poisoning, including accidental overdose, accounted for 104,200 of the adverse drug reactions. Notably, accidental overdose can occur even when the staff are monitoring the patient because signs of accidental overdose may be difficult to recognize.

Concern about overmedication hampers the administration of analgesia in multiple medical settings including postoperatively and in the use of patient-controlled analgesic (PCA) systems. If accidental drug overdoses could be avoided or quickly reversed, more effective pain management could be implemented without fear of patient overdose and the quality of patient care would increase.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a block diagram of a system for tracking and reversing accidental overdose.

FIG. 2 is a schematic view of an example physical embodiment of the system of FIG. 1.

FIG. 3 is a schematic view of a first embodiment of a control unit that can be used in the system of FIG. 2.

FIG. 4 is a schematic view of a second embodiment of a control unit that can be used in the system of FIG. 2.

FIG. 5 is a flow diagram of an embodiment of a method for tracking and reversing an accidental overdose.

### DETAILED DESCRIPTION

As described above, concern about overmedication hampers the administration of analgesia but more effective pain management could be provided if such overdoses could be avoided or quickly reversed. Disclosed herein are systems and methods that can be used for such a purpose. In some embodiments, the system monitors a patient to whom an analgesic medication is being administered and, if one or more patient parameters fall below an established threshold, alerts the attending staff and/or a physician as to this circumstance so that actions can be taken to reverse a possible overdose. In some embodiments, the patient parameter is the patient's respiration rate and the alert is generated when the patient's rate of respiration falls below a predetermined level. In further embodiments, the system can administer a reversal agent to the patient that counteracts the effects of the analgesic medication to reverse the overdose.

In the following disclosure, various specific embodiments are described. It is to be understood that those embodiments are example implementations of the disclosed inventions and that alternative embodiments are possible. All such embodiments are intended to fall within the scope of this disclosure.

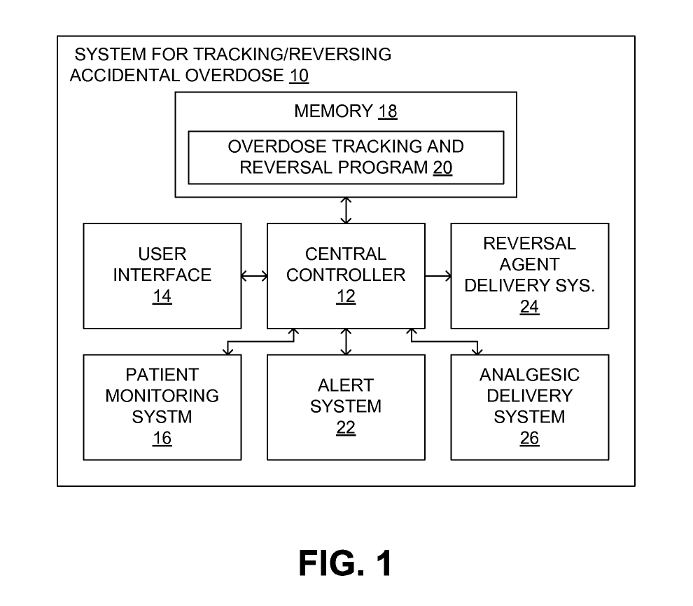

FIG. 1 illustrates an example system 10 for tracking and reversing accidental overdose. As shown in the figure, the system 10 comprises a central controller 12 that is in electrical communication with multiple other components of the system. In the illustrated example, these other components include a user interface 14, a patient monitoring system 16, memory 18 (i.e., a non-transitory computer-readable medium) that stores an overdose tracking and reversal program 20, an alert system 22, and a reversal agent delivery system 24. As is further shown in FIG. 1, the system 10 can optionally include an analgesic delivery system 26.

The central controller 12 can, for example, comprise a microcontroller in the form of a microchip. In some embodiments, the central controller 12 can be specifically programmed for controlling the system 10. In such a case, the memory 18 can comprise firmware that is comprised by the central controller 12. Regardless, the overdose tracking and reversal program 20 comprises code (i.e., computer logic) that includes the instructions that are used to control operation of the various components of the system 10 relative to sensed parameters.

The user interface 14 comprises the device or devices through which a user, such as a staff member or physician, operates the system 10. For example, the user interface 14 can be used to power up and initialize the system and activate patient monitoring. In addition, the user interface 14 can be used to set various system parameters. For example, the user interface 14 can be used to set patient parameter thresholds (e.g., respiration levels) at which certain system actions are to occur and establish what actions are to be performed when those thresholds are crossed (e.g., activating an alarm and/or activating the reversal agent delivery system 24). The user interface 14 can comprise one or more buttons, keys, or displays that facilitate interaction with the system 10. As described below, the user interface 14 can, in some embodiments, comprise a touch-sensitive display.

The patient monitoring system 16 is the part of the system 10 that monitors the one or more patient parameters that will be used to determine when one or more actions are to be taken by the system. The patient monitoring system 16 can, in some embodiments, monitor a patient vital sign, such as patient respiration rate. The respiration rate can be monitored in several ways. In some embodiments, the patient monitoring system 16 comprises a belt that is wrapped around the patient's torso that detects expansion and contraction of the torso that is indicative of patient breathing. In other embodiments, the patient monitoring system 16 comprises a sensor that measures blood pCO₂ or pO₂, which also provide an indication of patient respiration. In other embodiments, multiple patient parameters are simultaneously monitored. For example, the patient monitoring system 16 can comprise a sensor that measures respiration rate and a sensor, such as a pulse oximeter, that measures the patient's O₂ saturation. These are but a few examples of patient parameters that can be monitored.

The alert system 22 can be used to issue warnings to the patient and/or alarms to the staff/physician when certain patient parameters are detected using the patient monitoring system 16. As is discussed in greater detail below, the alert system 22 can activate an alarm for the attending staff and/or a physician if one or more patient parameters fall below the established threshold. In some cases, the alert system 22 can also activate a warning for the patient that signals to the patient that, unless he or she performs some affirmative action within a given timeframe to communicate to the system that he or she is alert (and therefore not in fact overdosing), the alarm will be activated and steps may be performed to reverse the effects of the analgesic medication that is being administered.

The reversal agent delivery system 24 is used to administer a reversal agent that is intended to reverse the effects of the analgesic medication being administered in circumstances in which the system 10 determines that, based upon the monitored patient parameters, an overdose is occurring. As is described below in greater detail, the reversal agent delivery system 24 can comprise one or more reservoirs of reversal agent that can be delivered as needed to the patient via an intravenous (IV) line. In some cases, the reversal agent can be administered after the alarm is activated or contemporaneous with alarm activation.

In cases in which the analgesic delivery system 26 is included in the system 10, it can be used to deliver the analgesic medication to the patient. In some embodiments, the analgesic delivery system 26 comprises a patient-controlled analgesic (PCA) system in which a basal amount of medication (set by the user via the user interface 14) is continuously administered to the patient and the patient can intermittently increase the amount of medication that is delivered, for example, by depressing a button on a patient controller. When the system 10 determines that an overdose is occurring, the system can operate to interrupt delivery of further analgesic medication to the patient.

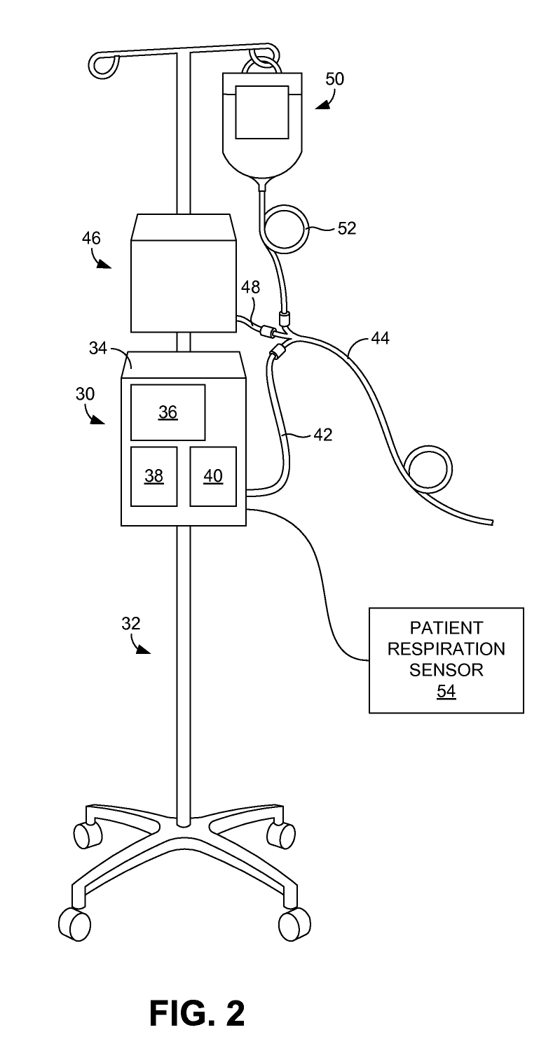

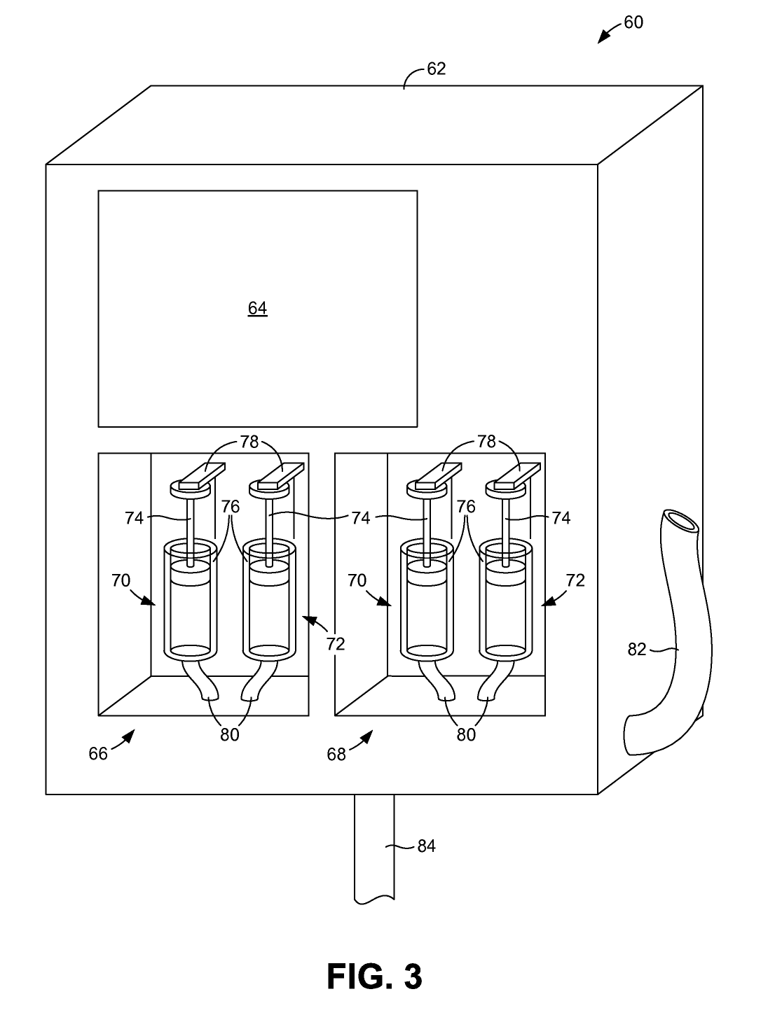

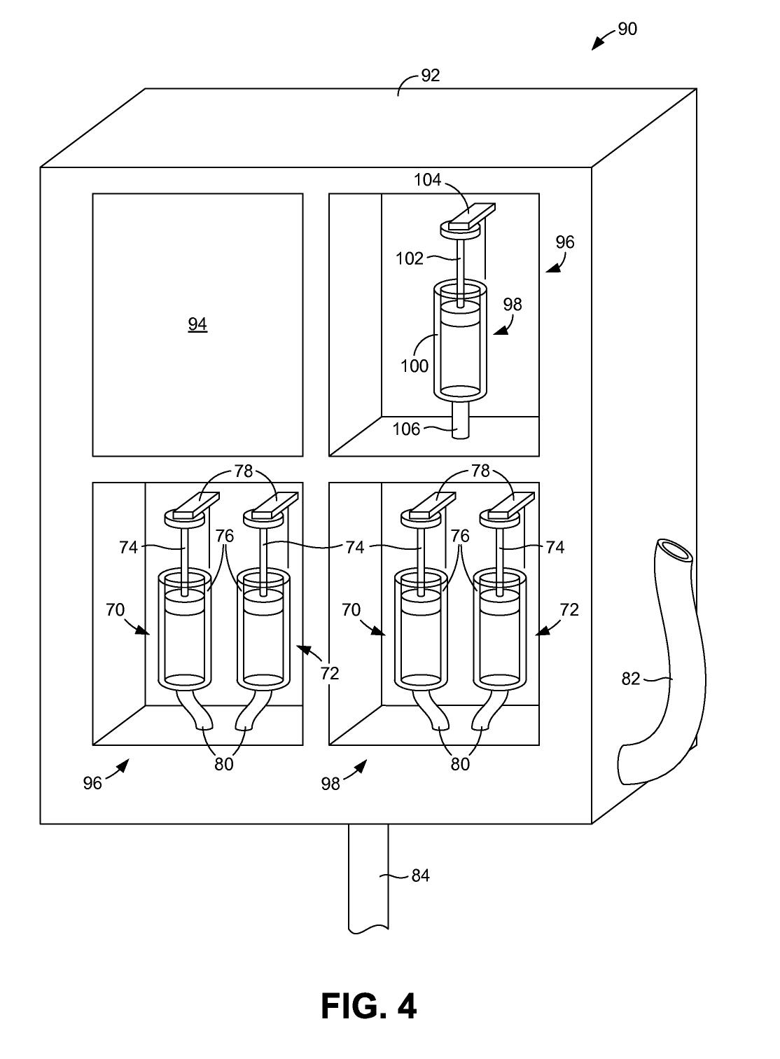

FIG. 2 illustrates an example physical embodiment of the system 10 shown in FIG. 1. As indicated in FIG. 2, a control unit 30, which can comprise the central controller 12, user interface 14, memory 18, alert system 22, and the reversal agent delivery system 24, is shown mounted to an IV pole 32 that can be placed next to a bed in which the patient resides. In the example of FIG. 2, the control unit 30 comprises a housing 34 that supports a touch-sensitive display 36, which serves as the user interface, and first and second cabinets 38 and 40 that, as described in relation to FIGS. 3 and 4, form part of the reversal agent delivery system. Extending from the housing 34 is a fluid line 42 that connects to an IV line 44 that is in fluid communication with the patient's intravenous system.

With further reference to FIG. 2, also mounted to the IV pole 32 is an independent analgesic delivery system 46 that also comprises a fluid line 48 that is connected to the patient IV line 44. The system 46 and line 48 can be used to administer an analgesic medication, such as an opioid-based analgesic medication, to the patient. Hanging from the top end of the IV pole 32 is an IV bag 50 that also comprises a fluid line 52 that connects to the patient IV line 44. The bag 50 and line 52 can be used to administer an IV fluid, such as a saline-based fluid, to the patient.

As is further shown in FIG. 2, a patient respiration sensor 54 is connected to the control unit 30. As described above, the respiration sensor 54 can, for example, comprise a belt that wraps around the patient's torso and detects expansion and contraction that is indicative of patient breathing.

FIG. 3 illustrates a first example embodiment of a control unit 60 that can be used in a system for tracking and reversing accidental overdose and that, for example, can be used as to the control unit 30 shown in FIG. 2. As indicated in FIG. 3, the control unit 60 includes a housing 62 that supports a touch-sensitive display 64 that can serve as the user interface for the system. Below the display 64 are first and second cabinets 66 and 68 that form part of the reversal agent delivery system. In the illustrated embodiment, each cabinet contains two liquid reservoirs in the form of first and second syringes 70 and 72. These syringes 70, 72 each contain a liquid that can be administered to the patient if it is determined that an overdose is occurring.

In some embodiments, the first syringe 70 contains an IV fluid, such as a saline-based or dextrose-based solution, and the second syringe 72 contains a reversal agent, such as an opioid antagonist (e.g., naxalone), that can counteract the effects of an analgesic medication that is being administered to the patient. In some embodiments, each syringe 72 contains a single dose of reversal agent that can be administered. In such a case, the two syringes 72 comprise two independent doses of reversal agent that can be separately administered. For example, if the patient parameters indicate that an overdose is occurring, a first dose can be administered to the patient. If after the passage of a predetermined amount of time the patient parameters have not returned to a level above the threshold, the second dose can be administered.

In the example of FIG. 3, the reversal agent is administered by depressing a plunger 74 into the barrel 76 of the syringe 72. This can be achieved using an appropriate actuation mechanism. In the example of FIG. 3, the actuation mechanism includes a lever 78 associated with each of the syringes 70, 72 that can be displaced downward by an internal piston or other driver (not visible) to push the plunger 74 into its barrel 76. When the plunger 74 is driven into the barrel 76, its liquid contents exit the barrel through a fluid line 80 that is in fluid communication with a further fluid line 82 that connects to the patient IV line. When reversal agent is to be administered, it can be delivered from its syringe 72 and the IV fluid can then be delivered from its syringe 70 to drive the reversal agent along IV line to ensure it reaches the patient.

As is further shown in FIG. 3, a power cable 84 that is used to provide power to the control unit 60 can extend from the housing 62. In some embodiments, the control unit 60 can also house an internal power source, such as a battery, to ensure the system will operate even when power is no longer supplied to the control unit by the power cable 84.

Although not illustrated in FIG. 3, each cabinet 66, 68 can include a door that can be locked to prevent unauthorized access to the syringes 70, 72 or other internal components of the control unit 60.

FIG. 4 illustrates a second example embodiment of a control unit 90 that can be used in a system for tracking and reversing accidental overdose. The control unit 90 is similar in many ways to the control unit 60 shown in FIG. 3. Accordingly, the control unit 90 includes a housing 92 that supports a touch-sensitive display 94 and first and second cabinets 96 and 98 that form part of the reversal agent delivery system. Like the cabinets 66, 68, the cabinets 96, 98 each include first and second syringes 70 and 72 that can contain an IV fluid and a reversal agent, respectively. These liquids can be administered in the same way as that described above in relation to FIG. 3.

In the embodiment of FIG. 4, however, the control unit 90 further includes a third cabinet 96 that forms part of an analgesic delivery system, which can be a PCA system. The third cabinet 96 contains a further reservoir in the form of a syringe 98 that comprises a barrel 100 filled with an analgesic medication that can be delivered to the patient when the plunger 102 is depressed by the lever 104. As with the other syringes 70, 72, the contents of the syringe flow out through a fluid line 106 that is in fluid communication with the further fluid line 82, which is connected to the patient IV line. When, as shown in FIG. 4, the control unit 90 incorporates the analgesic delivery system, the system can further operate to interrupt administration of analgesic medication when the monitored patient parameters indicate that an overdose may occur. It is noted that, in embodiments in which the system does not incorporate the analgesic delivery system, the system may not operate to interrupt the administration of analgesic medication. In such cases, the reversing agent dosage can be selected to ensure that it will be enough to counteract the effects of the analgesic medication even if it is continued to be administered.

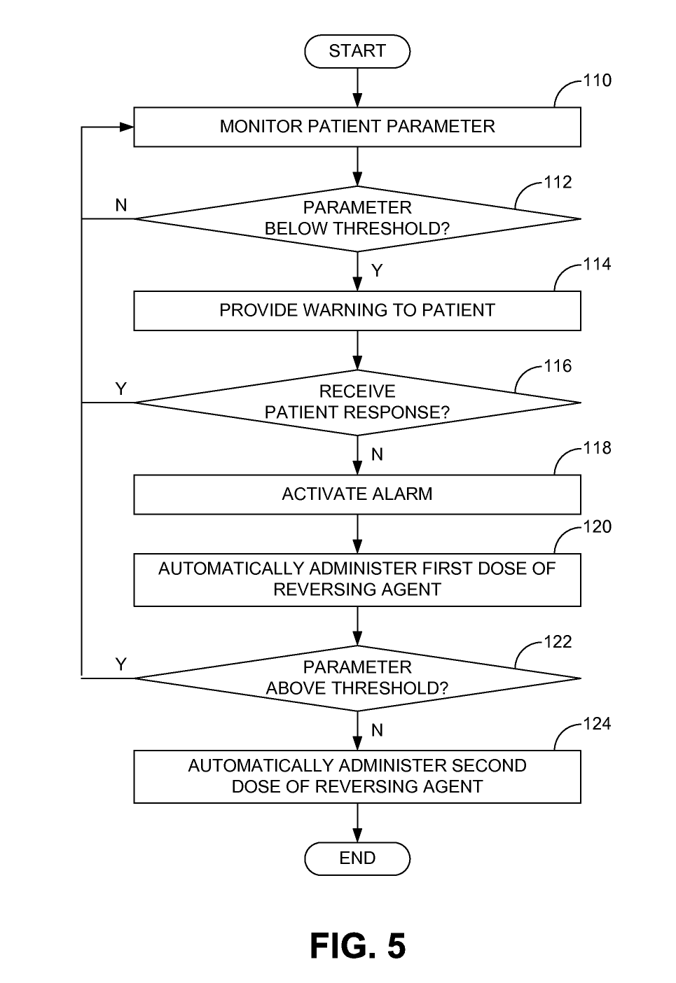

FIG. 5 is a flow chart that describes an example method for tracking and reversing an accidental overdose, which can be performed using one or more of the systems described above. In this example, it is assumed that the patient is being administered an analgesic medication that could induce an overdose.

Beginning with block 110, at least one patient parameter is measured that provides an indication as to whether or not the patient is overdosing. As described above, the parameter can, for example, be the patient respiration rate. Notably, however, any patient parameter that provides an indication of possible overdose can be monitored.

Referring next to decision block 112, it is determined whether or not the patient parameter has fallen below a given threshold. The threshold can, in some embodiments, be set by the user with the user interface. As an example, if the patient parameter is the respiration rate, the threshold can be set to be 10 breaths per minute. Regardless, if the parameter does not fall below the threshold, the likelihood of an overdose condition is unlikely and no action need be taken. In such a case, flow returns to block 110 and the monitoring continues.

If the patient parameter falls below the threshold, however, flow continues to block 114 at which a warning can be provided to the patient, for example using the alert system 22 described above in relation to FIG. 1. The warning can be provided to the patient prior to taking other actions because, if the patient is conscious and able to respond to the warming, it is unlikely that he or she is overdosing. In such a case, it would be preferable not to administer it to the patient. In some embodiments, the warning can comprise one or more of an alarm tone and a verbal warning. Regardless of its nature, the warning signals the patient that he or she must complete a given task to convey that he or she is alert to prevent administration of the reversing agent. When the warning is a verbal warning, it can explain what task must be performed. The task to be performed to prevent the administration of the reversal agent can also vary. In one embodiment, the patient must press a button on a controller, such as a PCA controller. In other embodiments, the task can comprise providing an appropriate verbal response that can be recognized by the system.

Once the warning has been provided to the patient, flow continues to decision block 116. With reference to this block, if an appropriate patient response is received, the patient is not deemed to be overdoing. In such a case, flow returns again to block 110. If, on the other hand, an appropriate response is not received within a predetermined period of time, flow continues to block 118 at which an alarm is activated. Unlike the patient warning, the alarm is intended for the attending staff and/or a physician and alerts them to the fact that the patient may be overdosing. As with the patient warning, the alarm can take various forms. In some embodiments, the alarm is an audible alarm that is emitted by the control unit positioned near the patient. In other embodiments, the alarm further comprises an electronic signal or message that is transmitted to one or more electronic devices, such as a hospital computer and/or one or more pagers or smart phones. In such a case, the appropriate persons can be alerted even if they are not proximate to the patient and cannot hear the audible alarm.

In addition to activating the alarm, the system can, as indicated in block 120, administer a first dose of reversing agent to the patient in the manner described above in an effort to reverse the overdose that the patient appears to be having. Some time after this agent has been administered, it can be determined whether or not the patient parameter has risen above the threshold, as indicated in block 122. If so, the overdose has been reversed and flow can return to block 110. If not, however, the first dose of reversing agent was not enough to reverse the overdose. In this case, a second dose of reversing agent can be administered, as indicated in block 124.

In some embodiments, various events that occur during operation of the system can be logged, which may comprise storing the events in system memory. For example, each occurrence of the patient parameter falling below the established threshold, each warning that is issued, each alarm that is activated, and each time reversing agent is administered can be logged to generate a record of patient care.

Claimed are:

1. A system for tracking and reversing accidental overdose because of administration of an analgesic medication, the system comprising:
a patient monitoring system configured to monitor a patient parameter that provides an indication as to whether or not the patient is overdosing;
an alert system configured to activate when the patient parameter falls below an established threshold; and
a reversal agent delivery system configured to automatically administer a reversing agent that counteracts the effects of the analgesic medication when the patient parameter falls below the established threshold.

2. The system of claim 1, wherein the patient monitoring system is configured to monitor a patient parameter indicative of patient respiration.

3. The system of claim 1, wherein the patient monitoring system is configured to monitor the patient's respiration rate.

4. The system of claim 3, wherein the patient monitoring system comprises a belt that wraps around the torso of the patient.

5. The system of claim 1, wherein the alert system is configured to activate an alarm that alerts an attending staff member or physician that the patient parameter has fallen below the established threshold.

6. The system of claim 5, wherein the alert system is further configured to provide a warning to the patient that a reversal agent will be administrated to the patient unless the patient performs a required task that communicates to the system that the patient is alert.

7. The system of claim 1, wherein the reversal agent delivery system is configured to automatically administer an opioid antagonist to the patient.

8. The system of claim 1, wherein the reversal agent delivery system comprises a reservoir that contains a single dose of reversal agent.

9. The system of claim 8, wherein the reservoir comprises a syringe from which the reversal agent can be expelled.

10. The system of claim 8, wherein the reversal agent delivery system further comprises a reservoir that contains an intravenous liquid that can be administered after the reversal agent is administered to ensure the reversal agent reaches the patient.

11. The system of claim 1, wherein the reversal agent delivery system comprises two reservoirs, wherein each contains a single dose of reversal agent that can be separately administered to the patient.

12. The system of claim 1, further comprising an analgesic delivery system configured to administer the analgesic medication to the patient.

13. The system of claim 12, wherein the system is configured to interrupt administration of analgesic medication to the patient when the patient parameter falls below an established threshold.

14. A non-transitory computer-readable medium that stores an overdose tracking and reversing program comprising:
logic configured to determine if a patient parameter has fallen below an established threshold because of administration of an analgesic medication;
logic configured to activate an alert when the patient parameter falls below the established threshold; and
logic configured to activate a reversal agent delivery system to automatically administer a reversing agent that counteracts the effects of the analgesic medication when the patient parameter falls below the established threshold.

15. The computer-readable medium of claim 14, wherein the logic configured to determine is configured to determine if the patient's respiration rate has fallen below an established threshold.

16. A method for tracking and reversing accidental overdose, the method comprising:
monitoring a patient parameter to determine whether or not the patient is overdosing;
if the patient parameter falls below an established threshold, activating an alert and automatically administering a dose of reversal agent to the patient that counteracts the effects of the analgesic medication.

17. The method of claim 16, wherein monitoring comprises monitoring the patient's respiration rate.

18. The method of claim 16, wherein activating an alert comprises activating an alarm that alerts an attending staff member or physician that the patient parameter has fallen below the established threshold.

19. The method of claim 16, wherein activating an alert comprises providing a warning to the patient that a reversal agent will be administrated to the patient unless the patient performs a required task that communicates to the system that the patient is alert.

20. The method of claim 16, further comprising automatically administering a second dose of reversal agent to the patient if the patient parameter does not rise above the established threshold after the administration of the first dose.
